(19) United States
(12) Patent Application Publication
(10) Pub. No.: US 2025/0281311 A1
(43) Pub. Date: Sep. 11, 2025

(54) MECHANISM AND METHOD FOR DONNING ORTHOTIC DEVICE TO PROSTHESIS, LIMB, OR JOINT

(71) Applicant: Icarus Medical, LLC, Charlottesville, VA (US)

(72) Inventors: David T. Johnson, Charlottesville, VA (US); Evan Eckersley, Charlottesville, VA (US); Philip Miller, Charlottesville, VA (US); George Miroulis, Virginia Beach, VA (US)

(21) Appl. No.: 19/074,062

(22) Filed: Mar. 7, 2025

Related U.S. Application Data
(60) Provisional application No. 63/562,674, filed on Mar. 7, 2024.

(51) Int. Cl. A61F 2/78; A61F 5/01

(52) U.S. Cl. CPC A61F 2/78; A61F 5/0123; A61F 2002/7862

(57) ABSTRACT

System for connecting an orthotic device to a prosthesis or limb.

### CROSS-REFERENCE TO RELATED APPLICATION

The present application relies on the disclosures of and claims priority to and the benefit of the filing date of U.S. application No. 63/562,674, filed Mar. 7, 2024. The disclosures of that application are hereby incorporated by reference herein in their entireties.

### BACKGROUND OF THE INVENTION

The present invention is an improved mechanism and method to connect an orthotic device to a prosthesis or limb that is convenient, easy to don and doff, and yet secure. The device features an upper frame and cuff that is fit to the patient's limb, and a lower frame with a clamping cuff that attaches via a suspension system to the prosthesis or stump. An adjustable tensioning system allows for the suspension system to incrementally increase in tightness around the socket and provide the necessary force to prevent migration from the desired position. The suspension system comprises at least two components that swing open on one side for a donning and doffing method that does not require stepping through the device. Both lower components are mechanically joined to the upper frame coplanar to a body joint. This combination ensures that articulation of the body joint is in congruence with articulation of the orthosis.

The invention includes reference to embodiments of prosthetic attachment mechanism(s), fabrication methods, integration with orthoses, and swing assist adjustable tensioning hinge technology according to U.S. patent application Ser. No. 17/902,683 entitled Unloading Knee-Ankle-Foot Orthotic Apparatus with Conforming and Distracting Hinge, which is hereby incorporated by reference herein in its entirety.

The invention includes reference to embodiments of load distribution elements for use in prosthetic and orthotic attachment mechanisms as described in U.S. patent application Ser. No. 17/864,675 entitled Unloading Knee Brace Apparatus, which is hereby incorporated by reference herein in its entirety.

The invention includes reference to embodiments of closure mechanisms, suspension systems, adjustable tensioning systems, and adjustment dials as described in U.S. patents application Ser. Nos. 18/075,203 and 18/387,433, each entitled Adjustable Tensioning Device, which are hereby incorporated by reference herein in their entireties.

The invention includes reference to embodiments of orthotic and prosthetic devices with motors and sensors, as described in U.S. patent application Ser. No. 17/074,571 entitled Assistive Orthotic Device with Motors and Sensors, which is hereby incorporated by reference herein in its entirety.

There are approximately 185,000 amputations performed in the United States per year and an estimated 1 million performed worldwide. The total population of amputees in the US is estimated to be 2.1 million people. Most amputations are due to vascular degeneration with trauma being the second leading cause. Lower limb amputations are more common than upper limb amputations—likely because lower limbs are more likely to be subject to diseases such as cancer and diabetes.

Not all amputees are fitted with a prosthetic limb; functional prosthetics require retraining the wearer on how to compensate for the loss of muscle and balance. However if the patient is reasonably healthy, capable of maintaining the site of the amputation and properly cleaning/maintaining the prosthetic limb, and have the need, a prosthetic limb may be fitted by a specialist. The skin covering the stump may be desensitized while healing to facilitate wearing the prosthetic for long periods of time. Often, prosthetics and especially lower-limb prosthetics lack dynamic functionality. However, conditions like contracture, arthritis, or joint instability are common in patients who wear prosthetics. Therefore, a system to improve function and stability of a range of custom prosthetics is required.

Prosthetics are nearly always custom-fabricated. A prosthetist typically begins by taking a plaster mold of the limb remnant. This enables the prosthetist to create a socket that matches the patient's physiology. A pylon (the central support) is attached to the socket and is often made of metal or carbon fiber for strength. Depending on the use, functional elements are attached to the pylon—for example, a foot plate for a lower leg prosthetic. Aesthetic elements may also be added to mimic the look of the missing limb. A liner is added to the inner surface of the socket to provide a soft, clean interface between the prosthetic and the limb remnant. Finally, the prosthetic is fitted with a suspension system. The suspension system is the combination of buckles, straps, fitting, suction, etc. that hold the prosthetic to the limb remnant

The suspension system is very important to the proper function of the prosthetic. The suspension system must account for the intended use. For example, if a prosthetic arm will be used to lift household objects like a paint bucket the suspension system must be strong and secure enough to stabilize the prosthetic arm to the limb when lifting 10 or more pounds. Likewise, the suspension system for a prosthetic lower leg used for a wearer who wishes to run a marathon must absorb the repeated shocks of foot fall and not chafe when dealing with heat and perspiration over a period of several hours.

The art of designing successful suspension systems depends on the intended use of the prosthetic and the habits of the amputee. The suspension system must be secure but it also needs to be easy to don and doff, easy to use, easy to clean, comfortable and lightweight. There are many variations of suspension systems in the art that strive to find a better solution for these—sometimes conflicting—requirements.

While many such suspension systems to affix the prosthesis to the residual limb have been contemplated, there is little prior art relating to mechanisms that connect an orthosis or other functional device to the prosthesis. Amputees, for example below knee (BK) amputees, suffer from knee instability, patellar instability and dislocation, extensor mechanism weakness, impaired gait, and knee arthritis due to trauma. For non-amputee patients with these symptoms, a knee orthosis may be prescribed. However, challenges in connecting a functional orthosis (or brace) to a prosthetic limb prevent amputees from realizing the benefits that these devices can provide. Traditional strapping and suspension mechanisms are ineffective in preventing brace migration due to the rigid surface and unique contours of a prosthetic limb.

### DESCRIPTION OF RELATED ART

U.S. Pat. No. 9,872,790 assigned to BOA Technologies™ describes a dial based system for tightening two sides of a prosthetic socket to the wearer's limb remnant. In one embodiment, a flap is attached to the main body of the prosthetic by a hinge. In another embodiment, the circumference of the prosthetic shell is larger than the circumference of the limb so that the prosthetic shell can wrap around itself. Twisting a dial tightens a lace which draws the prosthetic shell and/or flap tight to the limb. The dial is advantageous in adjusting the tightness of fit and can be adjusted throughout the day as the wearer's limb remnant swells of shrinks, but the invention herein is an improvement thereon.

U.S. Pat. Nos. 9,956,094, 10,918,502, and 11,083,602 assigned to Click Medical™ describe variations of an article or methods of manufacturing an article which is a prosthetic shell with movable panels and a dial. Twisting the dial tightens a lace which compresses the panels against the limb remnant. One patent describes a method to use liquid resin for at least part of the shell and a system of guides such that after curing there is a channel for the lace. Another patent describes using the lace to drive an actuator to tighten the shell. The third patent describes the preferred geometry of the panels being located in cutouts (“ports ”) of the prosthetic socket shell. But the invention herein is an improvement thereon.

References WO 2009029191 and WO 2009029191 assigned to Ossur™ describes a socket shell fabricated from a resilient material. Applying tension to a tensioning element compresses the resilient material which conforms to the shape of the limb remnant. U.S. Pat. No. 7,488,349 assigned to Ossur™ describes a flexible socket shell with a vertical cutout that compresses to the limb remnant when buckles are tightened thereby drawing the sides of the cutout towards each other. U.S. Pat. No. 7,105,122 assigned to Ossur™ describes an air bladder situated between a hard socket shell and the limb. Applying pressure to the air bladder fills the void between the limb and socket wall. But the invention herein is an improvement thereon.

U.S. Pat. No. 6,991,657 assigned to J. B. Price, Jr. describes a semi-rigid socket shell with a cutaway. Tightening straps around the shell compresses which then conforms to the limb remnant. U.S. Pat. No. 11,844,667 assigned to J. Johnson describes a preferably 3D printed socket with apertures through which loose inserts can apply compression to the limb remnant when a compression cord is retracted. But the invention herein is an improvement thereon.

U.S. Pat. No. 11,642,233 assigned to Ossur™ describes an interchangeable fixture to couple a socket with a suspension system thereby allowing for a customized socket with an off-the-shelf suspension system, but the invention herein is an improvement thereon.

As seen from the examples cited above, most of the related art is focused on improved methods of attaching a functional prosthesis to a limb. However, there is a need for a better mechanism and method of attaching an orthotic to a prosthesis, which is described herein.

It is just as likely that amputees suffer from joint problems as non-amputees—especially from trauma caused amputations. A person with a lower leg amputation may very well have a knee joint problem. Knee orthotics are designed to strap to a person's upper and lower leg. The strapping systems are designed for specific leg shapes and tissue. For example, the lower straps of a knee orthotic are meant to wrap around the calf of the wearer's leg which bulge outwards and then taper into towards the knee and ankle and are conformable. A lower limb prosthesis is likely to be smaller in circumference than a typical leg with a more or less constant diameter and fabricated from a hard, relatively slippery material (e.g. carbon fiber).

Traditional orthotic devices are not designed for partial limb prostheses. There is a need for a better method to clamp orthotic devices to prosthetic limbs and limb remnants. The clamping mechanisms must ensure a secure fit, must be easy to don and doff, must be strong and lightweight, and must provide a way to adjust the clamping force to the prosthetic. Additionally, there is a need for a modular system to provide dynamic, adjustable functionality to patients with a range of custom prostheses.

### SUMMARY OF THE INVENTION

According to the current invention, it is possible to design and manufacture clamping systems specific to the needs of orthotic-to-prosthetic coupling. Because almost all prostheses have at least some custom-fabricated components, an off-the-shelf clamping solution will be a compromise at best. Additionally, while patients with prosthetic limbs, such as below knee (BK) amputees, may benefit from the stability, gait assistance, and security provided by orthotic devices, a secure connection to the orthosis and prosthesis is required to ensure proper function.

The present invention includes several embodiments which achieve effective orthotic-prosthetic coupling through combinations of several elements and methods.

One method includes the ability to scan the prosthesis and render its shape as an STL file that allows the practitioner to import it into a computer program where a customized clamping component can be designed. A combination of 3D scanning software and hardware, automated design algorithms, CAD software, and additive or subtractive manufacturing provide a method to match a custom surface to the surface of the prosthesis or limb. Such 3D mapping methods allow for the contouring of the orthotic device's surface to the prosthesis to provide a form-fit and avoid migration.

One mechanism described is an adjustable tensioning system, which allows the user to attach, detach, open, close, tighten and/or loosen the suspension system via an adjustment mechanism coupled to a lace or cable, and further connected to the device frame and the clamp mechanism. Adjustment mechanisms include dials, allowing the user to firmly secure the orthosis' frame to the prosthesis or limb.

In aspects, the interfaces of the orthotic suspension system include high-friction or adhesive materials, including glue, epoxies, hook and loop, or cat-tongue fabric. In other aspects, the interface may include rubbers, meshes, foams, or other compliant materials that secure the prosthesis while the suspension system is tightened.

In other aspects, pegs, slots, protrusions or interlocking surfaces may provide connection between the orthosis and prosthesis.

Each element independently, or in combination, may achieve the desired output of a secure yet reversible prosthetic-orthotic coupling. The invention further describes functional orthoses comprising various mechanisms and functions including those to stabilize the joint, unload the joint to provide pain relief, provide knee extension assistance, improve gait, distract the joint, or prevent migration of the prosthesis. The invention also describes functional prosthesis and prostheses (devices that are a combination of a prosthetic and an orthotic device.

### BRIEF DESCRIPTION OF THE DRAWINGS

The accompanying drawings illustrate certain aspects of some of the embodiments of the present invention and should not be used to limit or define the invention. Together with the written description the drawings serve to explain certain principles of the invention

FIG. 1 An orthogonal view of one aspect of the present invention.

FIGS. 2a-2c An orthogonal view of a hinge and tab mechanism.

FIGS. 3a-3b A side view of knee orthotic clamped to a prosthetic limb.

FIGS. 4a-4c An orthogonal view of a prosthetic and corresponding panel.

FIG. 5A cross-sectional view of the prosthetic and panel shown in FIGS. 4a-4c.

FIGS. 6a and 6b Posterior view of a prosthetic with a mounting system for an orthotic.

### DETAILED DESCRIPTION

An aspect of the current invention includes methods, software, and additive manufacturing processes to achieve an accurate, conforming surface between the suspension system or orthosis frame and the prosthesis or limb. By scanning the prosthesis (for example by using a mobile 3D scanning application or 3D scanning hardware) and capturing it as an STL file, local valleys and peaks can be accurately mapped on the surface of the prosthesis. Corresponding peaks and valleys are preferentially mapped on the clamping surface of the orthosis. When the clamping surface is affixed to the prosthesis, the matching of the peaks-to-valleys and valleys-to-peaks between the two parts helps lock them securely in the desired position relative to each other. Traditional methods of matching one surface to another are done by covering one surface with a dye, mating or rubbing the surfaces together, and removing material where the dye transferred to the mating surface. This method transfers the location of the peaks of one surface to another but isn't efficient in capturing the valleys. By contrast, scanning software may employ photogrammetry, LIDAR, TrueDepth, or other technologies used to capture and compile 2D or 3D data to develop 3D renderings of a surface, for example as a point-cloud data set. The surface, represented by a series of points or splines, can be accurately represented in CAD software.

Such 3D renderings and data sets can be transferred to other applications that allow the manufacturer to perform computer based analysis such as Finite Element Analysis. Thus the part can be designed to exceed a minimal safety requirement but without resorting to excessive over-engineering. The computer analysis can be adjusted to accurately reflect the specific use case (e.g., the weight of the wearer—and thus the impulse force applied to the joint, the length of the limb—the lever arm, and so forth). It is not cost effective or timely to do similar analysis using traditional methods of manufacture leading to manufacturers to heavily over-engineer their products which can result in heavier, larger, and more cumbersome clamping methods.

Combining these technologies according to the current invention, it is possible to make a strong, lightweight clamping mechanism to securely join an orthosis to a custom prosthesis that has been tailored to the specific needs of the wearer. The ability to match the surface topology of the orthosis with the mating surface of the prosthesis is particularly important because it enables the use of engineering rigid or semi-rigid materials in the clamping interface instead of less strong, less secure compliant materials. Compliant materials allow an orthosis to be affixed to a prosthesis in a less than ideal location or in the wrong orientation. The inventions described herein are safer, stronger, and more secure than the prior art.

Whereas past technology mainly was designed to secure a prosthetic socket to a limb remnant (which can be compressed and whose shape/volume may change during the day/over time), the inventions described herein are specially designed to secure an orthotic to a prosthesis which is typical unyielding and whose shape—while varying from one iteration to another—remains constant for each iteration.

For purposes of an example only, without limitation of the scope of the invention described herein which will apply to other orthotic/prosthetic combinations, consider a patella femoral (PF) unloading knee orthosis and a below the knee (BK) prosthesis equipped with a carbon fiber blade footplate: If the prosthesis is designed for walking and particularly for running, the pylon may be a carbon fiber or titanium tube which may be an inch (˜25 mm) or less in diameter. The average male calf is 5 inches (˜125 mm) in diameter. PF unloading braces (such as the Ascender™ manufactured by Icarus Medical™ of Charlottesville, VA) can unload 40 lbs (˜18 kg) from the knee joint by transferring forces to the back of the calf and thigh which aid the wearer in straightening their leg. In practice, it is not possible to secure the tibial straps of a knee brace designed to be strapped to the wearer's calf to a 1 inch pylon and still have effective use of both the prosthesis and the knee brace. There is a need for a custom clamping system.

A scan of the prosthesis is used to generate a 3D STL of the socket, pylon, and foot blade. The STL file is then imported into a CAD program and overlaid with a CAD representation of the knee brace. This enables proper orientation and position of the orthotic and the prosthesis with relation to each other. The location and size of the clamping mechanism is determined by considering the security needed (use case) and the geometry of the prosthetic socket. For a PF knee orthotic, the clamping interface is preferably located between 6-10 inches (150-250 mm) below the knee joint to generate the torque required for unloading the knee joint. When this is not possible, a larger surface area clamping band may be required.

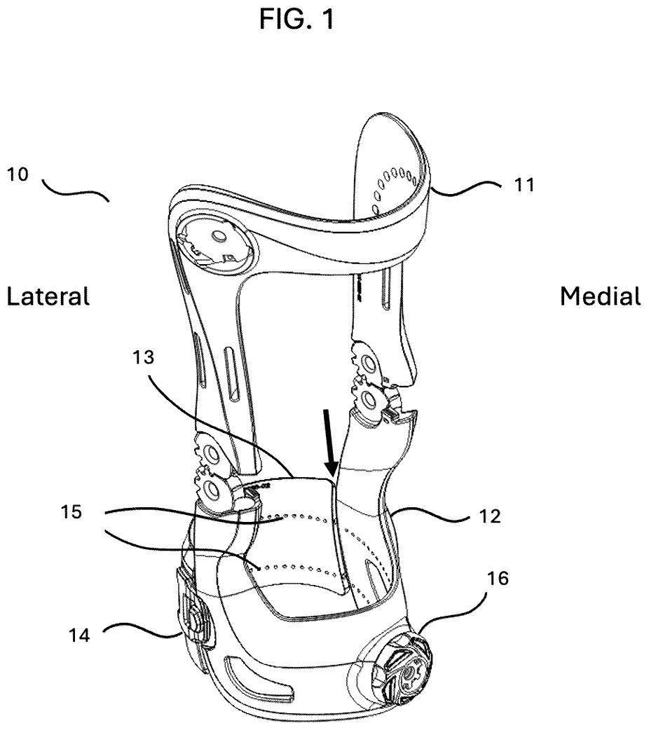

FIG. 1 depicts one embodiment (10) of the present invention incorporated into a patella femoral osteoarthritis (PFOA) unloading knee orthosis for the right leg. The clamping interface is divided into an anterior component (12) and posterior (13) component (otherwise referred to an upper frame and a lower frame). It is intended that the knee orthotic will be donned and doffed after the prosthesis is donned. In aspects, the division between the anterior and posterior components should not include any undercuts that would prevent bringing the anterior and posterior components from closing around the prosthesis (not shown). The anterior component of the clamping mechanism is affixed or preferably integral with the frame (11) of the PFOA knee orthosis. The posterior component of the clamping mechanism is separate from the anterior component (or lower frame) and coupled by a flexible tension component such as a braid, lace, cable, string, and the like (not shown). The path of the flexible tension component can be inferred by following the lines of dots (15) which are an artifact of the manufacturing process (holes to remove unincorporated material from the 3D printing process). A tensioning adjustment mechanism (16) (e.g., a dial with spool) is affixed to the anterior component integrated with the knee orthotic. The flexible tension component is operationally coupled to the tensioning adjustment mechanism and guided through the posterior component of the clamping mechanism such that applying tension forces via the tensioning adjustment mechanism draws the anterior and posterior components of the clamping mechanism into contact with the prosthesis securing the orthotic to the prosthesis.

In one embodiment, the flexible tension component is looped around a stud, flange, ridge, or the like on one side of the anterior component of the clamping mechanism wherein the ends of the flexible tension component pass through channels, guides, or the like in the posterior component of the clamping mechanism, and continue to other side of the posterior component where they couple with the tensioning adjustment mechanism. In the embodiment illustrated in FIG. 1, one end of the flexible tension component is attached to the tensioning adjustment mechanism (16). The flexible tension component follows a path along the anterior component, crosses the separation between the anterior and posterior component, and continues in a path along the posterior component. The flexible tension component couples with a buckle (14) and returns in a path along in the posterior component, crosses the separation between the posterior and anterior component, and continues along a path in the anterior component where the second end of the flexible tension component attaches to the tensioning adjustment mechanism.

In aspects, the path of the flexible tension component may be defined by a tunnel, an elongated guide, a plurality of guides, channel, valley, linear distributions of eyelets, openings, posts, pins, and the like, or combinations thereof.

As shown in FIG. 1, the medial side of the posterior component is connected to the anterior component with the flexible tension component. This allows the posterior component to hingably open along the axis indicated by the arrow so that the wearer can don the knee orthosis while wearing a prosthesis.

In another embodiment, one end of the flexible tension component is fixed to the anterior component and the other end of the flexible tension component is coupled to the tensioning adjustment mechanism. Alternatively, both ends of the flexible tensioning component are coupled to the tensioning adjustment mechanism. Alternatively, the path of the flexible tensioning component can zigzag multiple times between the anterior and posterior components to strengthen the coupling connection between the two components.

In another embodiment, one side of the posterior component is coupled to the anterior component by a hinge, an elastic strip, a cloth band, wire mesh, polymeric living hinge, flexible element, or the like that allows the posterior component to close about the prosthesis similar to a door.

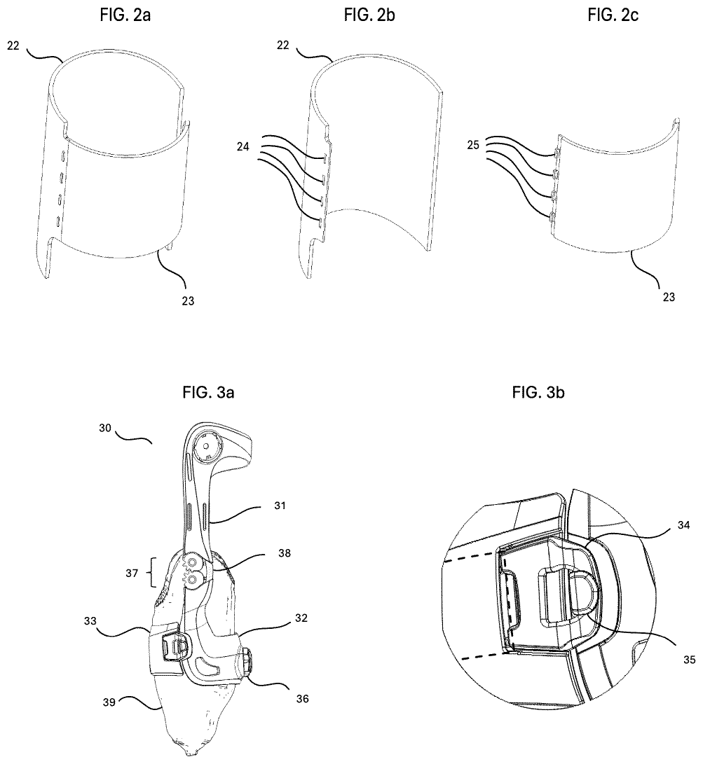

In another embodiment, tabs and slots, lips and shelves, bead and cove, clips and anchors and the like are arrayed on corresponding sides of the anterior and posterior components such that the components are coupled by one component slotting hingably into the other component. FIG. 2a shows an aspect of the present invention where the posterior component (23) is hingably attached to the anterior component (22) with tabs and slots. FIG. 2b shows an array of slots (24) positioned along one edge of the anterior component. FIG. 2c shows the corresponding array of tabs (25) positioned along one edge of the posterior component. The second edge of the posterior component is drawn towards the second edge of the anterior component by tightening an adjustable tensioning mechanism (not shown) thereby clamping the posterior and anterior components to the prosthesis (not shown).

Magnets, rails, and other physical and/or visual guides may be used to help the user align the posterior and anterior components into the proper position for clamping to the prosthesis.

FIG. 3a shows a side view of the PFOA knee orthosis (30) shown in FIG. 1 clamped to a lower leg prosthesis (39). The internal faces of anterior component (32) and posterior component (33) are designed to comply with the peaks and valleys of the external face of the prosthesis. When the adjustable tensioning mechanism (36) is used to apply tension to the flexible tensioning component, the posterior and anterior components clamp shut around the prosthesis. In this embodiment, the unloading PFOA knee orthosis has a polycentric hinge (37). The hinge cap used to connect the upper frame (31) with the anterior component (32) has been removed to show the location of the energy storage element (38) that generates the unloading force when tensioned.

FIG. 3b is an enlargement of the buckle and post feature shown in FIG. 3a. The dotted line in FIG. 3b shows the path of the flexible tension component with relation to the buckle (34) and the posterior component. The post (35) is integral or operationally coupled with the anterior component. When donning the orthosis, the tensioning adjustment mechanism is set to low or no tension to provide slack to the flexible tension component. In aspects, the buckle is operationally coupled to the posterior component by the flexible tension component. The slack in the flexible tension component allows the buckle to be positioned over the post. Applying tension to the flexible tension component with the tensioning adjustment mechanism secures the buckle to the post and clamps the posterior and anterior component to the prosthesis.

As shown in FIG. 1 and FIG. 3a the flexible tension component draws the free end of the posterior component towards the anterior component of the clamping mechanism thereby securing the orthotic to the prosthesis. In an alternative embodiment, the tensioning adjustment mechanism is affixed to the posterior component instead of the anterior component. In yet another embodiment, multiple tensioning adjustment mechanisms and multiple flexible tensioning components are employed which enables the clamping force to be customized at different positions between the posterior and anterior components of the clamping mechanism.

In another embodiment the clamping interface is divided into an anterior component and a plurality of separate posterior components. A flexible tension component is laced between the anterior and posterior components and is operationally coupled to a tensioning adjustment mechanism. The path of the flexible tension component is not continuous circumferentially around the orthotic but is interrupted which provides an ‘opening’ between the anterior and posterior or between one and an adjacent posterior components. In the ‘open’ configuration, the orthotic is fitted around the prosthesis. The posterior components are wrapped around the prosthesis and the ‘opening’ is operationally closed (e.g., by looping the flexible tension element over a ridge, catch, flange, etc.). Applying tension via the tensioning adjustment mechanism clamps the anterior and plurality of posterior components of the clamping mechanism to the prosthesis. The posterior components may be hinged to an adjacent posterior component and or hinged to the anterior component.

In another embodiment, the clamping interface is divided into a plurality of fingers that are connected flexibly at their base to the orthotic, for example, as the petals of a flower are connected to the eye of the flower. A flexible tension element wraps circumferentially around or through the fingers. The flexible tension element is operationally coupled to a tensioning adjustment mechanism such that when tension is applied to the flexible tension element the fingers clamp against the prosthesis.

In another embodiment, the posterior component is composed of a compliant material, such as a thermoplastic, rubber or foam, wherein tensioning of the suspension system causes the posterior portion to form to and secure the prosthetic interface.

In another embodiment, the clamping interface is comprised of a series of woven or coiled strands (such as in a chinese finger trap), wherein when tension is applied to the clamping interface, the circumferential compressive force securing the prosthesis is increased.

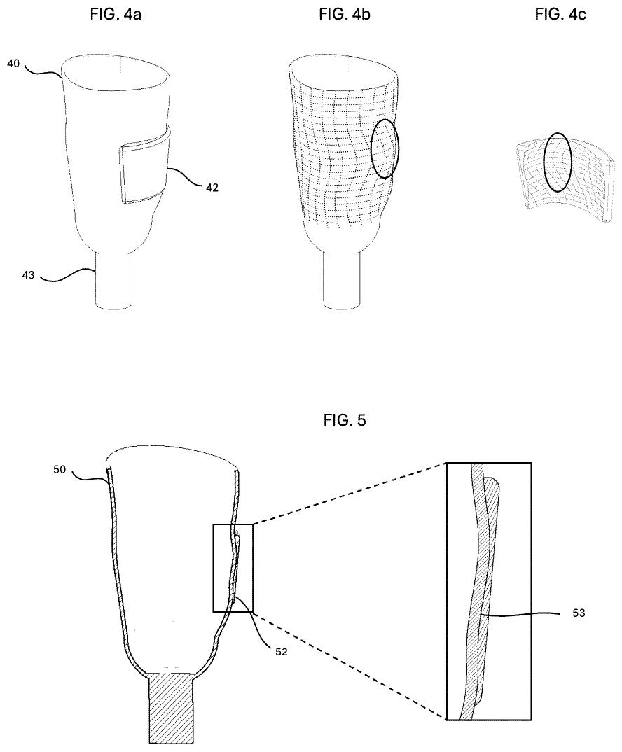

FIG. 4a is an orthogonal view of a lower limb prosthesis (40) with an integrated pylon (43) and a corresponding panel (42) that matches the surface topography of the prosthesis. The panel is representative of the anterior component and/or the posterior component shown in FIG. 1. FIG. 4b is a view of the prosthesis (40) with isolines superimposed. The curvature of the isolines indicate regions where the surface rises or dips. FIG. 4c shows the isolines on the inner surface of the corresponding panel (42). The oval in FIG. 4b shows a region on the surface that has a local peak. The oval in FIG. 4c shows the corresponding region on the panel with a local valley. FIG. 5 is a cross-sectional view of the prosthesis and panel shown in FIG. 4a. The enlargement clearly shows how the inner surface of the panel matches the topography of the outer surface of the prosthesis. A close match of the inner and outer surfaces improves the security of clamping the orthotic to the prosthesis.

In aspects, a compliant material can be placed at the interface (53) between the panel and the prosthesis to aid in the matching of peaks to valleys. In aspects, a high friction material can be placed at the interface. In aspects, a soft material (softer than the hardness of the prosthesis) can be placed at the interface to prevent the panel from creating surface scratches on the prosthesis. In aspects, a material that combines some or all of the properties described above can be placed at the interface. Materials placed at the interface may be attached primarily to the prosthesis, attached primarily to the panel, or one material may be attached to the prosthesis and a second material (either the same or different from the first material) may be attached to the panel. In aspects, material placed at the interface may be a discrete component and not attached to either the prosthesis or the panel but placed between the panel and prosthesis during the process of donning. The material or materials positioned between the panel and the prosthesis may completely or partially cover the interface between the panel and the prosthesis.

Traditional prosthetic sockets and pylons are not designed for specific attachment means for orthotics and other accessories. The clamping mechanisms described above overcome this limitation. While a standard interface may never be practical since each prosthetic socket is tailored to the wearer's limb remnant shape and length it is possible to add features that facilitate future clamping needs.

For example, when fabricating the socket for a prosthetic limb, the prosthetist could incorporate dimples around the socket proactively. A dimple would not protrude from the prosthesis and therefore would not catch on clothing and other objects. Likewise, the dimple could be relatively shallow with a gradual curvature to minimize unappealing aesthetics and the collection of dirt/grime. If, in the future, an orthotic, an accessory, or the like needed to be clamped to the prosthesis, the dimples could be used as anchor points. In other words, the dimples would be “valleys” that were purposely incorporated into the prosthesis.

Due to the custom nature of prosthetic sockets, the prosthesis would still need to be 3D scanned to generate an STL of topology of the prosthesis. As before, after aligning the prosthesis and the orthosis in a computer, a clamping interface with corresponding valleys for the prosthesis peaks and peaks for the prosthesis valleys is generated. The embodiments described above could all be employed with the added benefit that the dimples would provide extra clamping surface area and thus better security. In addition, the dimples would act as reference elements that would help quickly and correctly align the orthotic to the prosthesis.

In the previous clamping mechanism embodiments, the clamping components are preferably fabricated from a rigid or semi-rigid material to maximize the locking force generated by the peak and valley topology. By introducing features into the prosthesis (such as the dimples) the security of the clamping interface can be improved such that shock absorbing materials (which are typically compliant) can also be used. In practice, features such as dimples could be cut into an existing prosthesis but these would likely be less aesthetically pleasing than features that were introduced during fabrication. Also, the integrity of the shell of the socket may be compromised by material removal after fabrication.

Other features in addition or instead of dimples are envisioned. An annular channel around the outside of the prosthesis is one such feature. An annular channel has the advantage that—if it were the main clamping feature—the orthotic could rotate about the axis of the prosthesis. (If the peaks and valleys of the topology of the rest of the prosthesis were of large enough magnitude they would prohibit such rotation though.) Contrarily, if no rotation was desired a wavy annular channel, or a channel with some asymmetric feature could be employed.

For extra security, a female feature like a channel with an undercut could be fashioned in the prosthesis. In addition to tightening the clamping mechanism of the orthotic to the prosthesis, applying tension to the flexible tensioning component could be employed to move elements into the undercuts which would literally lock the orthotic to the prosthesis.

In the examples above, the female features are located in the prosthesis and the corresponding male features are located in the clamping surface of the orthotic. The reverse is possible (although care would need to be taken that the male protrusions on the prosthesis didn't catch clothing, objects, or have sharp edges). It is also possible to glue elements onto an existing prosthesis. While not as elegant as incorporating features during the manufacturing of the prosthesis, gluing clamping features allows for modifications after-the-fact and may be preferable when machining into the prosthetic shell is not recommended.

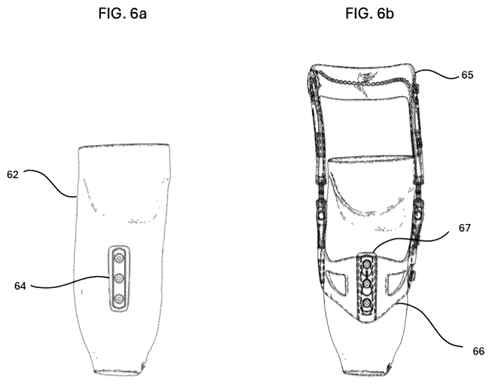

FIG. 6a and FIG. 6b illustrate another aspect for connecting an orthotic to a below the knee prosthesis. The location of the clamping mechanism is estimated on the prosthesis. A plurality of protrusions (62) (e.g., buttons) are bonded to the posterior outer surface of the prosthesis (62). Suitable bonding means could be epoxies, glues, hot melt adhesives, screws, bolts, rivets, and the like. The prosthesis is scanned and rendered as an STL and imported into a computer program. The orthotic is superimposed in its proper orientation and position. A receiver (67) which engages with protrusions (62) is generated and integrated into the lower frame element (66) of the orthotic. The top frame element (65) which straps to the wearer's upper leg is connected to the lower frame element. Optionally, a finite element analysis is run to test the proposed design for safety, fitness for use, etc. If necessary, modifications are made to the clamping mechanism geometry based on the results of the FEA. The clamping mechanism is integrated directly with the orthotic or designed such that it can be affixed to an orthotic. The design is converted to a suitable computer file and is fabricated (for example by 3D printing). In aspects, the mechanical connection provided by the protrusions (62) and the receiver (67) is sufficient to secure the orthotic to the prosthesis. In other aspects, an additional clamping attachment method such as the panel shown in FIG. 5 with a tensioning adjustment mechanism to clamp the panel to the prosthesis can be used for greater security. In aspects, the orthosis may include a locking or cap component to secure the interface between slots and protrusions.

The inventions described herein are especially applicable to the coupling of an orthotic to a functional prosthesis. Non-functional prostheses are typically human-form replications that are donned by amputees for aesthetic purposes but do not replicate the function of the missing limb. The methods described herein could be used to attach an orthotic to non-functional prosthetic limbs although the need for an orthotic for the joint of a non-functional limb let alone the need for a secure connection between an orthotic and a non-functional prosthesis is likely vanishingly small.

In some instances, several prostheses are meant to attach to a wearer's socket. Instead of having two separate prosthetic limbs (one for day-to-day life and one specifically for swimming, for example) occasionally a socket is configured to accept different pylons and functional attachments. If the wearer needs an elbow orthosis to limit range of motion, for example, they may equally need it while swimming and during day-to-day life. In this manner, the orthosis preferably would couple to the socket only and not rely on coupling to the entire prosthesis.

The embodiments described above are equally suitable for securing an orthotic to a prosthetic socket without the pylon, etc.

In some instances the orthotic and the prosthesis are the same device (or so integrated it is as if they are one device). The clamping mechanisms described above could be modified to clamp to the limb remnant. As before, a scan of the limb is used to generate a topological map of the limb and imported into a computer program. A computer representation of the orthosis/prosthesis is overlaid on the limb for proper positioning. A clamping interface is consequently generated that is the negative of the limb topology. The clamping mechanism is integrated into the orthosis/prosthesis. Optionally, computer analysis is performed on the computer file to simulate performance and the computer file is modified as needed. The file is rendered into a suitable computer file and fabricated. The materials used would still be engineering materials but additionally foam, padding, liners and the like would be needed for the limb/socket interface.

In aspects, the suspension system may be lined with compressible foam, padding, elastomeric material, or other material which has a high coefficient of friction to prevent migration on the limb or prosthesis, and/or may offer padding between the two components.

In aspects, one or more components may be provided as a kit. In the example of the Ascender™ knee brace paired with a BK prosthesis, the kit may include two hinges, elastomeric elements, an adjustment mechanism, and a suspension system. A certified prosthetist may incorporate one or more of the kit components into the fabrication of a lower limb prosthesis, and include vertical portions or a top cuff, which may be removable by the wearer. The kit would provide for an extension assist capability tunable to the needs of the individual wearer.

In aspects, the extension assist system may be manufactured continuously, for example with 3D printing.

In aspects, the upper portion or frame, lower anterior portion or frame, and/or lower posterior portion, can be manufactured continuously. In aspects, articulating hinges may be comprised of flexible or elastomeric regions of a continuously manufactured device.

In embodiments where a KO is combined with a prosthesis, a rotation joint may be incorporated to allow extension assist in the sagittal plane, but also to allow for free rotation about the mechanical axis of the joint. One version of the rotation joint may be built into vertical struts, or at the interface of the suspension system itself, allowing the prosthesis to rotate about a partially and/or approximately cylindrical relative to the upper portion of the limb. The joint may also be entirely rigid, or such that some flexibility is allowed. The prosthesis may be attached to the KO with typical attachment mechanisms known in the art of prosthesis fabrication, using a snap-lock, dovetail system, pin-lock, or rotation lock, for example.

In aspects, the suspension system, prosthetic or orthotic portion may comprise a guide element built into the prosthetic or orthotic structure directly. For example, a channel or tunnel could be created during the composite layup of a prosthesis that could be used as a guide element for the linking element. In other embodiments, a tube or other structure that defines the path of the linking element could be incorporated into the composite layup. In yet other embodiments, a sacrificial or temporary material (e.g., a wax tube, a PTFE cord, etc.) could be incorporated during the composite layup to define the guide element in the structure, but would be removed from the article once it had set.

In aspects the device further comprises a linking element, which is capable of connecting an energy storage element (e.g. an elastomer or spring) to an adjustable module and/or the structure of the prosthesis or orthosis. The adjustment module allows the wearer to tailor the unloading force to their needs while the prosthesis or orthosis is being worn. The adjustment module, for example a dial, may improve the fit of the clamp, fasten the clamp, tighten straps, or adjust tension within a tensioning element.

In one embodiment, the function of the brace orthosis can be built into the prosthesis, and so the Icarus™ tensioning system (referenced above) is integrated within the prosthetic socket. The socket can have a compression system similar to that of the Quorum™ prosthetic socket that will compress the limb.

Another embodiment can involve a clamping mechanism that conceals an internal lace system and involves a latch-type mechanism that allows parts of the latch to slide on themselves to secure one part of the orthosis to another part of the orthosis. On the other side of the latch mechanism is a type of gate that allows one part of the orthosis to articulate or rotate around the other part, so that the wearer can easily don and doff the orthosis from their prosthesis without sliding the orthosis over the prosthesis. The gate-type mechanism can either be permanently attached to the other part of the orthosis, or it can be removable. It is also possible to have one part of the clamp attached to the prosthesis.

The orthosis and clamping or suspension system described herein can be used to secure a range of devices to the prosthesis, not only an orthosis, such as microprocessor and/or control system to help assist or control the prosthesis or joint. A battery pack may also be attached, as well as motors and/or sensors. This device would have the capability to make any type of orthosis assistive, for any type of limb. Attachment mechanisms described could be used. Elastic members and/or tensioning elements could be used in-line with the assistive mechanism that controls the prosthesis. A prosthesis comprising an upper and lower part with a joint in between that can assist extension, and/or resist flexion, and/or involve distraction forces, can be controlled with similar electronic microprocessor systems, and with or without a tensioning element in-line either directly or indirectly between the upper and lower portions of the prosthesis. This embodiment would be suitable for patients with significant mobility impairment, such as partial paralysis.

The orthosis and clamping or suspension system described herein can be used to secure an orthotic or prosthetic device to a limb or body part.

Other aspects include:

A closure device, orthosis, prosthesis or combinations thereof (collectively referred to as the device) comprising one or more tensioning elements and an adjustable tensioning mechanism, wherein the adjustable tensioning mechanism is coupled to the one or more tensioning elements, wherein the adjustable tensioning mechanism comprises an interface between the adjustable tensioning mechanism and a wearer, and may include a knob, slide, button, tab, digital screen, processor, controller, motor, microdrive, switch, pulley, block and tackle system, or lever, that the wearer can use to adjust the fit of the prosthetic closure, or generate a force around a region of the device, for example around a wearer's joint.

In aspects, the device further comprises one or more sensors that measure and monitor the position of the brace, wherein the one or more sensors are optionally capable of measuring and monitoring velocity or acceleration, wherein the position data, velocity data, or acceleration data, are used as input to a processor or monitoring system for the joint brace, and wherein the position data, velocity data, or acceleration data is used to instruct a motor or other tensioning system on the device to assist or support a joint by increasing or decreasing resistance in the device, or tension in the one or more tensioning elements.

In aspects, the device can have sensors, wherein the one or more sensors are capable of measuring and monitoring an amount of tension present in the joint brace or the one or more tensioning elements, or the amount of unloading force applied at a wearer's joint.

In aspects, the device can incorporate a digital signal, wherein the digital signal informs a wearer of the device regarding how much tension is present in the device or as a change in tension is recognized by the one or more sensors.

In aspects, the device can have sensors, wherein the one or more sensors are fabricated on or within the device. In aspects, the one or more sensors output a digital or electronic signal, and the one or more sensors connect to one or more lights or other indicator, including a viewing port, that indicate information about the device, including an amount of force or tension in the device.

In aspects, the current invention relates to an orthotic that clamps to a functional prosthetic device, wherein the orthotic supports a joint between the stump and the body, or wherein the orthotic simulates a missing joint beyond the stump.

In aspects, the current invention relates to an orthosis that clamps directly to a limb or non-functional stump cap/covering.

“Element” and “component” are used herein interchangeably.

While some embodiments of the current invention describe lower limb orthoses, prostheses, or prosthoses (prosthetic and orthotic device combinations), these embodiments are by example only. One skilled in the art will recognize that the disclosed features may apply to to upper extremity or upper body devices, including arm and hand prostheses, orthoses, and prosthoses. Embodiments of the current invention also apply to exoskeletons.

Other aspects and embodiments of the invention are described below.

In aspects, the suspension system, prosthesis, or orthosis comprise a system of pins, pegs, protrusions or buttons (or other male elements) that interlock with slots or female elements on another component. Such male components may be provided as a kit for fastening to the prosthesis with bolts, rivets, adhesives and the like for security and alignment with the orthotic device. Such a pin system may also allow the orthosis to wedge onto the prosthesis and lock into the pin system to prevent migration, especially for prosthesis that have a tapered shape. Migration can also be prevented by designing the orthosis to wedge against another feature of the prosthetic system. For example, the connecting modules may protrude from a socket and the orthosis can rest on features of the module to prevent migration.

In aspects, one embodiment of the device comprises only a single member of an orthosis combined with lace that is connected to the tensioning system, without the use of a second member and the force exerted would be from the lace or tensioning elements exerting a compressive force around the prosthesis.

In aspects, the orthosis and prosthesis are connected with a slidable locking system comprising slots or grooves.

In aspects, the suspension system further comprises elements to affix the prosthetic limb to a joint or body part, including straps, corsets, or closure mechanisms.

Although embodiments described herein refer to orthoses with suspension systems that can detach from the prosthesis, orthoses, such as the Icarus™ Medical Ascender knee brace, can be fixed to the prosthesis with bolts, rivets, and adhesives.

Another embodiment of the present invention comprises vertical slots or receptacles in the prosthetic limb, which may be added as a component. Vertical struts of the orthosis frame can slide into the vertical receptacles, and be connected with pins, latches, switches, or bolts. The position of the orthosis can be adjusted with a peg and slot system, and in aspects, the orthosis is capable of being rapidly detached from the prosthesis.

In embodiments, the orthotic-prosthetic combination is one continuously printed 3D printed device. In other aspects, the prosthetic and orthotic components are both printed separately. A modular hinge kit may be added, for example in a BK device, to enhance suspension, improve range of motion, produce extension assistance, or provide joint unloading. The upper cuff of the orthosis can be interchangeable with different sockets as an option.

One embodiment of the present invention can be used immediately post-amputation as an immediate post-operation prosthesis (IPOP). In this embodiment, the temporary device can attach a limb orthosis to the prosthesis in one of the ways described herein, or these can be integrated or combined into a single system that can be optionally 3D printed. An ideal setup is an IPOP that also has a high-torque extension assist like in the Ascender knee brace mechanism by Icarus Medical.

In one embodiment, the invention is a system for coupling an orthosis to a prosthesis comprising: a first member of an orthosis that partially conforms to a prosthesis; a second member of an orthosis that partially conforms to another part of the prosthesis; a tensioning system directly or indirectly connecting the first member with the second member; wherein the tensioning system comprises at least one adjustment mechanism and at least one flexible tensioning component; wherein the at least one adjustment mechanism is capable of producing a mechanical advantage in the at least one flexible tensioning component; wherein upon adjusting the adjustment mechanism causes, a tensioning force to be applied to the at least one flexible tensioning component; and wherein when the tensioning force is imparted from the orthosis members to the prosthesis, securing the orthosis is secured to the prosthesis.

In another embodiment, the invention is a lower limb prosthetic attachment system comprising: a first member having at least one vertical support comprising a rigid or semi rigid cuff (the top frame), a second member having at least one vertical support comprising a rigid or semi rigid cuff (orthosis part in front of prosthesis), and a third member comprising a rigid or semi rigid plate (orthosis part as back plate of prosthesis); wherein the first member is worn upon a first body part above the second member (top frame on thigh); wherein the second and third member are modeled and fixed around a lower limb prosthesis using a tensioning system, and wherein the connected second and third member are located below the first member (fit around prosthesis); wherein the first body part and the lower limb prosthesis are connected across a body joint, wherein the body joint articulates over a range of articulation; wherein the second and third members are fixed together around the prosthesis via a tensioning system, wherein a tightening of the tensioning system draws, pulls, or pushes, the second and third member together creating a compressive force around the lower limb prosthesis, wherein the tensioning system spans one or both gaps between the second and third members, wherein the second and third members can be disengaged and opened on one side of the one or more gaps without any disassembly; wherein a front and a back plate of the lower limb prosthesis are connected via a tensioning system that can be opened on one side; wherein a disengagement and engagement of the first and second tensioning member can be accomplished using lace and hook, rigid claws, buckling, strapping, or use of a doweled hinge (the different mechanisms that can go into the opening system); and wherein the first, second and third members are connected by a unicentric or polycentric hinge, wherein the unicentric or polycentric hinge articulates with the articulation of the body joint (the first, second, and third member are all attached via a gear hinge).

Another embodiment of the invention is a lower limb prosthesis comprising: a first member having at least one vertical support comprising a rigid or semi rigid cuff, wherein the first member is attached to a wearer's thigh, a second member having at least one vertical support comprising a rigid or semi rigid cuff located beneath the first member, and a third member having at least one peg comprising of a rigid or semi rigid plate; wherein the second member and the third member are modeled and fixed around a below knee amputation limb of the wearer, underneath the first member; wherein the first member and the second and third members are connected across a knee joint, wherein the knee joint articulates over a range of articulation; wherein the second and third members are connected together via a slot and key system; wherein the third member is fixed to the below knee amputation limb using adhesive, screws, bolts, or epoxy; wherein the first, second and third members are connected by a unicentric or polycentric hinge, wherein the unicentric or polycentric hinge articulates with the articulation of the knee joint.

One skilled in the art will recognize that the disclosed features may be used singularly, in any combination, or omitted based on the requirements and specifications of a given application or design. When an embodiment refers to “comprising” certain features, it is to be understood that the embodiments can alternatively “consist of” or “consist essentially of” any one or more of the features. Other embodiments of the invention will be apparent to those skilled in the art from consideration of the specification and practice of the invention.

It is noted that where a range of values is provided in this specification, each value between the upper and lower limits of that range is also specifically disclosed. The upper and lower limits of these smaller ranges may independently be included or excluded in the range as well. The singular forms “a,” “an,” and “the” include plural referents unless the context clearly dictates otherwise. It is intended that the specification and examples be considered as exemplary in nature and that variations that do not depart from the essence of the invention fall within the scope of the invention. Further, all the references cited in this disclosure are each individually incorporated by reference herein in their entireties and as such are intended to provide an efficient way of supplementing the enabling disclosure of this invention as well as provide background detailing the level of ordinary skill in the art.

As used herein, the term “about” refers to plus or minus 5 units (e.g., percentage) of the stated value.

Reference in the specification to, e.g., “some embodiments,” “an embodiment,” “one embodiment,” or “other embodiments” means that a particular feature, structure, or characteristic described in connection with the embodiments is included in at least some embodiments, but not necessarily all embodiments, of the inventions.

As used herein, the term “substantial” and “substantially” refers to what is easily recognizable to one of ordinary skill in the art.

It is to be understood that the phraseology and terminology employed herein is not to be construed as limiting and are for descriptive purpose only.

It is to be understood that the details set forth herein do not construe a limitation to an application of the invention.

Furthermore, it is to be understood that the invention can be carried out or practiced in various ways and that the invention can be implemented in embodiments other than the ones outlined in the description above.

1) A suspension system for coupling an orthosis to a prosthesis comprising:
a first rigid or semi rigid member of an orthosis positioned on a first side of a prosthesis;
a second rigid or semi rigid member of an orthosis positioned on a second side of a prosthesis; and
a tensioning system directly or indirectly connecting the first member with the second member;
wherein the tensioning system comprises at least one adjustment mechanism and at least one flexible tensioning component;
wherein upon adjusting the at least one adjustment mechanism, a tensioning force is applied to the at least one flexible tensioning component; and
wherein when a tensioning force is applied to the at least one flexible tensioning component, the first member and the second member are pulled, pushed, or drawn together, to secure or otherwise couple the orthosis to the prosthesis.

2) A system for coupling an orthosis to a prosthesis comprising:
a first member of an orthosis that partially conforms to a prosthesis;
a second member of an orthosis that partially conforms to another part of the prosthesis; and
a tensioning system directly or indirectly connecting the first member with the second member;
wherein the tensioning system comprises at least one adjustment mechanism and at least one flexible tensioning component;
wherein adjusting the adjustment mechanism causes a tensioning force to be applied to the at least one flexible tensioning component; and
wherein when the tensioning force is imparted from the orthosis members to the prosthesis, the orthosis is secured to the prosthesis.

3) An orthosis comprising:
a first member having at least one vertical support comprising a rigid or semi rigid cuff, a second member having at least one vertical support comprising a rigid or semi rigid cuff, wherein the second member is located under the first member and relative to one side of a prosthesis, and a third member comprising of a rigid or semi rigid plate located beside the second member, under the first member, and relative to a second side of the prosthesis;
wherein the first member is worn upon a first body part;
wherein the second and third members are modeled and fixed around the prosthesis;
wherein the first body part and a limb are connected across a body joint, wherein the body joint articulates over a range of articulation;
wherein the second and third members are fixed together around the prosthesis via a tensioning system, wherein a tightening of the tensioning system draws, pulls, or pushes the second and third members together thereby creating a compressive force around the prosthesis; and
wherein the first, second and third members are connected directly or indirectly by a hinge, wherein the hinge articulates with the articulation of the body joint.

4) The suspension system of claim 1, wherein the tensioning system is adjusted incrementally and wherein the adjustment mechanism is a rotary device.

5) The suspension system of claim 4, wherein the rotary device and tensioning system are capable of sustaining forces of greater than 25 lbs.

6) The suspension system of claim 1, wherein the first and/or second members are designed around a limb of a wearer of the device or the prosthesis, based on a three-dimensional scan of the limb of the wearer or the prosthesis.

7) The suspension system of claim 1, wherein the first and/or second members are modeled to conform with and/or contact over 60% of a surface area of a limb of the wearer of the device, the prosthesis, or both.

8) The orthosis of claim 3, wherein the second and third members house or include one or more elastic elements that span the body joint between (a) the first member and (b) the second and third members.

9) The orthosis of claim 8, wherein the one or more elastic element is attached to a rotary device that increases or decreases tension across the body joint.

10) The orthosis of claim 3, wherein the orthosis is for a knee, an ankle, a hip, an elbow, or a wrist.

11) The suspension system of claim 1, wherein the first member, the second member, or combinations thereof, comprise multiple panels, segments, or elements.

12) The suspension system of claim 1, wherein the first member is a distal frame of an orthotic device; wherein the first member is connected to a proximal frame of the orthotic device by a hinge; and wherein the hinge articulates with movement of a wearer's joint or body part.

13) The suspension system of claim 12, further comprising an energy storage element, wherein the energy storage element is capable of generating a force within, across, or between the wearer's joint or body part.

14) The suspension system of claim 12, further comprising a tensioning system, wherein the tensioning system comprises an adjustment mechanism coupled to a flexible tensioning element; and wherein the tensioning system is directly or indirectly connected to the proximal frame and the distal frame of the orthotic device.

15) A method of connecting an orthosis to a prosthesis, the method comprising:
obtaining a three-dimensional (“3D”) representation of outer surface topography of a prosthesis; and
super-imposing a 3D representation of an orthosis having an inner and an outer surface over the obtained topography of the prosthesis;
wherein at least a portion of the 3D representation of the orthosis is modified such that peaks in the inner surface of the orthosis correspond to valleys in the obtained topography of the prosthesis, or valleys in the inner surface of the orthosis correspond to peaks in the obtained topography of the prosthesis; and
wherein clamping the orthosis to the prosthesis creates a mechanical connection between the peaks and valleys that secures the prosthesis and orthosis such that the orthosis can function.

16) The method of claim 15, wherein the orthosis is clamped to the prosthesis by a panel that is drawn towards the prosthesis by a flexible tensioning component that is connected to an adjustable tensioning mechanism.

17) The method of claim 16, wherein the adjustable tensioning mechanism is a dial.

18) The method of claim 15, wherein the orthosis, the prosthesis, or both, are fabricated using additive or subtractive manufacturing.

19) The suspension system of claim 1, wherein the first member, the second member, or combinations thereof, further comprise a high-friction or adhesive material at an interface between the orthosis and the prosthesis.

20) The suspension system of claim 1, wherein the first member, the second member, or combinations thereof, further comprise a compliant material, wherein the complaint material deforms to match a surface of the prosthetic device when the tensioning system is tensioned.

21) The suspension system of claim 1, wherein the first member, the second member, or both, comprise one or more slots, which interlock with pegs, buttons, or protrusions on the prosthesis.

22) The suspension system of claim 1, wherein the suspension system is provided as a kit to be assembled with an orthosis, a prosthesis, or combinations thereof, by a clinical professional, a physician, or a wearer of the suspension system, the orthosis, the prosthesis, or combinations thereof.

23) The orthosis of claim 3, wherein at least one of the first member, the second member, or the third member, is fabricated continuously with the prosthesis.

24) The suspension system of claim 1, further comprising one or more tabs and slots, lips and shelves, bead and cove, clips and posts, or combinations thereof.

25) The suspension system of claim 1, wherein the prosthesis is a post-operative prosthesis or an immediate post-operative prosthesis.
